(19) United States
(12) Patent Application Publication
(10) Pub. No.: US 2026/0325288 A1
(43) Pub. Date: Oct. 22, 2026

(54) MEDICAL DEVICES AND METHOD FOR RECONSTRUCTING SEMILUNAR HEART VALVES

(71) Applicant: AVATAR MEDTECH CORPORATION, Mendoza (AR)

(72) Inventor: Ignacio Lugones, La Plata (AR)

(21) Appl. No.: 19/109,435

(22) PCT Filed: Sep. 5, 2023
(86) PCT No.: PCT/US2023/073462

§ 371 Date: Mar. 6, 2025

Related U.S. Application Data
(60) Provisional application No. 63/404,007, filed on Sep. 6, 2022.

(51) Int. Cl. A61F 2/24

(52) U.S. Cl. CPC A61F 2/2415; A61F 2220/0008; A61F 2240/005

(57) ABSTRACT

Medical devices and a method that utilizes them for reconstructing semilunar heart valves in patients with congenital or acquired cardiac diseases, using biologic or synthetic tissue, applicable to adult and pediatric patients.

### FIELD OF THE INVENTION

The present invention relates to medical devices and a method for reconstructing semilunar heart valves in patients with congenital or acquired cardiac diseases, using biologic or synthetic tissue. In particular, this invention is applicable to adult and pediatric patients.

### BACKGROUND OF THE INVENTION

Cardiovascular diseases are the leading cause of death worldwide. Among them, diseases of the semilunar heart valves-aortic and pulmonary-are very prevalent.

Aortic valve disease (congenital or acquired) affects more than seventy million people worldwide, and is characterized by valve stenosis and/or regurgitation. The most frequent etiology of aortic valve disease is degenerative disease, followed by rheumatic heart disease. Among congenital malformations, bicuspid aortic valve is the most common congenital heart defect.

Aortic stenosis is usually the consequence of degenerative remodeling in a normal tricuspid aortic valve or a congenitally abnormal valve, particularly a bicuspid aortic valve. The degenerative changes that lead to aortic stenosis, whether they occur in a bicuspid aortic valve or a normal tricuspid valve, are caused by an active remodeling process involving inflammation and calcification.

Although degenerative disease is the most common cause of aortic regurgitation, bicuspid aortic valve predisposes an individual to the development of aortic regurgitation, although less frequently than aortic stenosis. Rheumatic heart disease remains the second most common etiology of aortic regurgitation in developed countries and its most common etiology in developing countries.

It is considered that at least one third of patients with a bicuspid or unicomissural aortic valve will develop serious complications. Bicuspid or unicomissural aortic valves are responsible for 80-95% of cases of aortic valve disease detected early in life. This condition may be responsible for more deaths and morbidity than the combined effects of all other congenital heart defects.

The pulmonary valve is compromised in several congenital heart diseases, such as tetralogy of Fallot, pulmonary valve stenosis, or pulmonary atresia. Pulmonary valve insufficiency is very frequent in the evolution of patients operated on for Tetralogy of Fallot or truncus arteriosus.

Treatment of semilunar valve disease consists of valve repair or replacement, sometimes concurrently with repair of other heart defects. Open heart surgery is one of the treatments of choice, although in certain cases the percutaneous approach can be used. Conventional surgery is performed under general anesthesia, usually through an incision in the sternum and with the aid of a cardiopulmonary bypass machine. This machine performs the functions of the heart and lungs, oxygenating blood and distributing it throughout the body, allowing surgeons to stop the heart and repair it.

In case of having to perform a valve replacement, mechanical or biological prostheses can be used, the latter generally made of cow or pig tissue. Human pericardium can also be used for valve reconstruction.

According to the closest prior art, the heart valve replacement method comprises the steps of: cutting a portion of the pericardium; if the pericardium is human, place it in a substance that fixes it (if it is pericardium of another origin, it is already fixed); select a template or mold sized to fit the size needed for a patient based on previously made measurements; cut the parts of the pericardium to form the leaflets using the template as a guide; and suturing the leaflets to the artery to form the heart valve.

Ozaki Shigeyuki, in his patent JP5106019B2, discloses a template with perforations made in the form of leaflets of different sizes, which are used to draw and then cut an artificial or biological membrane in order to obtain leaflets for replacement.

The valve replacement method described by Ozaki targets aortic heart valves, is designed for adult patients, and involves the individual replacement of each leaflet. The size of each leaflet for replacement is calculated based on the size of the native leaflet to be replaced (measuring the intercommissural distance). When the native leaflets are asymmetrical, a fact that happens frequently, reconstruction using the method proposed by Ozaki is asymmetrical, leaving the point where the three leaflets meet not necessarily in the center of the artery. This determines an asymmetry in size between the three sinuses of Valsalva and an uneven distribution of pressure between them.

On the other hand, this method is not accurate for the replacement of bicuspid valves, since these valves have only two lines of insertion of the native leaflets, not three. Thus, the reconstruction of a valve with three leaflets cannot be done in a standardized way.

Despite various attempts to find an optimal method capable of reconstructing a heart valve, prior art methods still have shortcomings.

None of the prior art heart valve replacement techniques is designed for children. There are no replacement valves specifically sized for children, since due to their rapid growth, a prosthesis of these characteristics would be small very shortly after being implanted. In some children, who reach the size of adult cardiac structures, replacement valves can be used, but many of these valves result in the need for lifelong anticoagulant therapy. In addition, prosthetic valve replacement due to dysfunction or mismatch as the patient grows is very common and carries a high risk of morbidity and mortality, since the metal frame of the valve has fixed the native aortic annulus, preventing its growth.

Additionally, none of the proposed methods teaches how to automate some of the steps of the valve reconstruction method in order to reduce time, standardize the practice, improve precision and minimize the risks of surgery.

Consequently, there is a need for a method and medical devices that can be used in adults and children to replace a semilunar heart valve (aortic or pulmonary) with a new valve of the appropriate size for each patient, with the ability to remain competent while the child grows, without stopping the growth of the artery that contains it or requiring anticoagulation.

### SUMMARY OF THE INVENTION

In one aspect of the present invention, a method for the reconstruction of a semilunar heart valve is disclosed, comprising the steps of:
a) securing a portion of biological or synthetic tissue, which is extended using a first medical device, thereby firmly and tautly holding said portion of tissue, said first medical device comprising a shaping and marking part comprising two surfaces with an exterior configuration resembling that of a semilunar valve leaflet, designed to shape biological or synthetic tissue, said first surfaces having first grooves arranged to mark positions at which surgical sutures should be placed on the leaflet; and a fastening means for the shaping and marking part configured to maintain it in place, securing the tissue in a stretched and immobilized manner between the two surfaces;
b) generating a replacement leaflet from the tissue by cutting it around the shaping and marking part of the first medical device, which has a semilunar valve leaflet-shaped configuration;
c) placing sutures in the replacement leaflet at positions indicated by the first grooves of the first medical device;
d) opening a patient's artery and inserting a second medical device therein, said second medical device comprising a mold capable of indicating positions for sutures to be placed in order to insert the replacement leaflet;
e) passing the sutures, previously passed through the replacement leaflet at positions indicated by the first grooves of the first medical device, through positions indicated by the second medical device, passing through the artery wall;
f) suturing the nadir of the replacement leaflet to the wall of the artery to secure it;
g) removing the first medical device;
h) tying the sutures;
i) implanting a second and third replacement leaflet by repeating steps a) to h).

In another aspect of the invention, a first medical device applicable to the semilunar heart valve reconstruction method is provided, comprising:
a shaping and marking part comprising two surfaces with an exterior configuration resembling that of a semilunar heart valve leaflet, designed to shape biological or synthetic tissue, said first surfaces having first grooves arranged to mark positions at which surgical sutures should be placed on the replacement leaflet; and
a fastening means for the shaping and marking component configured to maintain it in place, securing the tissue in a stretched and immobilized manner between the two surfaces.

In yet another aspect, a second medical device applicable to the semilunar heart valve reconstruction method is provided, comprising a mold capable of indicating positions for surgical sutures to be placed in order to implant the replacement leaflets.

In still another aspect of the invention, a third medical device applicable to the semilunar valve reconstruction method is provided, wherein said device consists of a stitching clamp comprising a first surface at one of its tips with suitable fastening means to receive and hold suture needles, said fastening means being arranged in positions where the needles must traverse target tissue, a second surface at the other tip with at least one opening through which the needles pass upon exiting the tissue, and handles capable of bringing the tip surfaces closer, thereby passing a plurality of suture needles simultaneously through the target tissue.

### BRIEF DESCRIPTION OF THE DRAWINGS

The constructive characteristics and the advantages with respect to what is currently known in the art will be more evident in the present specification, considered together with the accompanying drawings, in which:

FIG. 1 illustrates a possible embodiment of the first medical device according to the present invention.

FIG. 2 illustrates a possible embodiment of the second medical device according to the present invention.

FIG. 3 illustrates another possible embodiment of the second medical device according to the present invention.

FIG. 4 illustrates a possible embodiment of the third medical device according to the present invention.

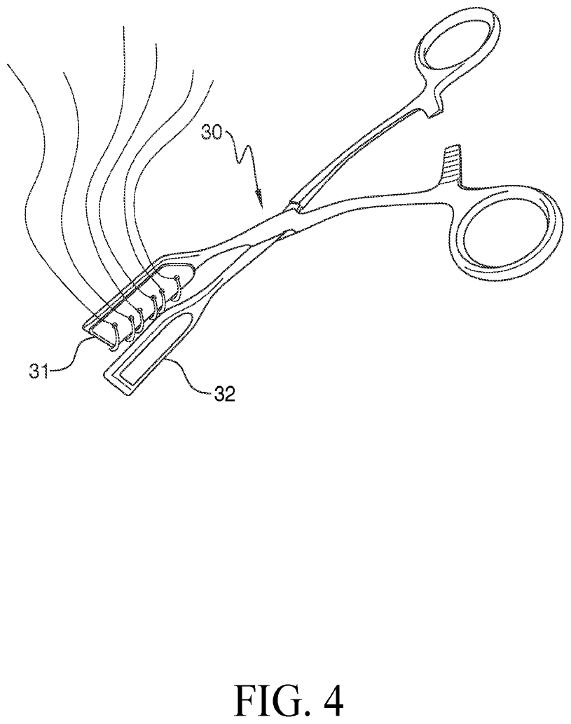

FIG. 5 is a schematic view of part of a patient's body in which the thorax and the pericardial sac are opened.

FIG. 6 is a schematic view of part of a patient's body in which the first medical device has been used to hold a portion of the pericardium.

FIG. 7 is a schematic view of part of a patient's body in which the replacement leaflet is being cut around the shaping and marking part of the first medical device.

FIG. 8 is a schematic view of the opening of a patient's artery, in this case the aorta.

FIG. 9 is a schematic view of a patient's artery with the second medical device in place.

FIG. 10 is a schematic view of a patient's artery, in this case the aorta, crossed by the sutures previously passed through the replacement leaflet in the positions indicated by the first grooves of the shaping and marking part of the first medical device in the positions indicated by the second grooves of the second medical device. The suture passed through the nadir of the replacement leaflet is anchored to the wall of the artery.

FIG. 11 is a schematic view of a patient's artery after the first and second medical devices have been removed.

FIG. 12 is a schematic view of a patient's artery with the replacement leaflet already sutured.

FIG. 13 is a schematic view of a patient's artery with all three replacement leaflets in place.

### DETAILED DESCRIPTION OF THE INVENTION

The invention will now be described in greater detail in connection with preferred embodiments, which should be considered only by way of example and not as limitations.

Normal semilunar heart valves are formed by three leaflets, with specific anatomical and histological characteristics. They are mobile and semi circularly attached to the root of the aorta or pulmonary artery 404. However, when the valve is pathological, the number of leaflets is variable: one (unicuspid), two (bicuspid), three (tricuspid) or four (quadricuspid). Moreover, the anatomical and histological features of the valve are pathological.

The semilunar heart valve reconstruction method of the present invention consists of the replacement of a diseased native valve having one, two, three or four leaflets by a symmetrical replacement valve with three leaflets 402, adapted to accommodate growth of the patient.

Replacement leaflets 402 are made from biological or synthetic tissue 400. Biological tissue 400 can be of human or animal origin. By way of example, and not as a limitation, biological source materials include pericardium, heart valves, arteries, and veins. The use of biological derivatives, such as extracellular matrices or collagen and heparin coatings, is also contemplated.

On the other hand, synthetic materials can comprise polymeric materials such as polytetrafluoroethylene (i.e. expanded polytetrafluoroethylene), polyethylene terephthalate, polyurethane, polyvinyl chloride, among others.

Preferably, the replacement leaflets 402 are made from autologous pericardium 400, that is, from the same patient.

In one aspect, the invention pertains to a first medical device 10 comprising:
a shaping and marking part 100 comprising two leaflet-shaped surfaces shaped to mold a biological or synthetic tissue 400, having these surfaces first grooves 102 arranged to mark positions in which surgical sutures 500 are to be placed on the leaflet 402, and

a fastening means 300 for the shaping and marking part 100, configured to hold the tissue 400 taut and trapped inside it in a secure and immobile fashion.

In one possible embodiment, the surfaces of the shaping and marking part 100 are flat. In another embodiment, the surfaces are concavely or convexly curved.

The surfaces of the shaping and marking part 100 can be made of plastic, although other possible materials, such as alloys of different metals, are contemplated.

The first grooves 102 are located on the surfaces of the shaping and marking part 100, preferably at positions corresponding to the commissures, adjacent margins and nadir of the leaflet 402.

In some embodiments, the first grooves 102 located in positions corresponding to the commissure and adjacent margins of the leaflet 402 are internal (located a few millimeters from the edge of the leaflet 402) and the first groove 102 corresponding to the nadir of the leaflet 402 (central groove) is external.

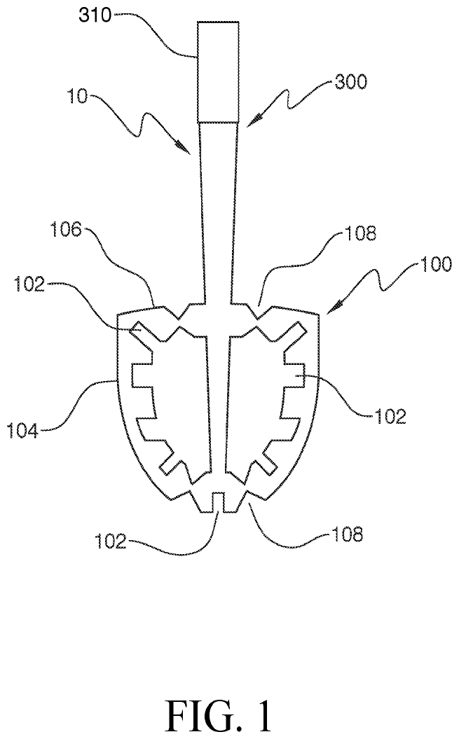

As illustrated in FIG. 1, the shaping and marking part 100 has a lower arched margin that corresponds to the suture margin 104, in which the sutures 500 that will fix the leaflet 402 to the artery 404 will be placed, and an upper margin, called the free margin 106, in which no suture will be placed. The latter corresponds to the mobile margin of the apposition zone that contacts the adjacent leaflet 402 to close the valve during diastole.

As can be seen, there are two types of first grooves 102. Some are wider, since two sutures 500 are placed there, the highest corresponding to the upper U and the lowest corresponding to the lower U. Therefore, of the first grooves 102, the upper and lower ones are narrower (only one needle passes through there), while the middle ones are wider because two sutures 500 pass through there.

Preferably, at least one of the surfaces of the shaping and marking part 100 has a roughness or fine teeth on at least one of its faces arranged to help hold the biological or synthetic tissue 400 when exerting pressure by means of the fastening means 300.

In some embodiments of the present invention, the surfaces of the shaping and marking part 100 are additionally provided with second grooves 108 designed to facilitate fracture and removal once the leaflet 402 has been placed in position and partially sutured within the aorta or pulmonary artery 404.

Preferably, there are four second grooves 108 arranged in such a way as to allow the surfaces to be fractured by separating their sides and central part. Thus, for example, the sides of the surfaces can be removed outwards and the central part towards the center and up.

The fastening means 300 may comprise two ratcheting clamp handles, with each handle attached to one of the surfaces of the shaping and marking part 100. Preferably, the fastening means 300 comprises box lock 302 and pin, shanks 304, ratchets 306 and ring handles 308. Each jaw 310 is attached to one surface of the shaping and marking part 100.

In other embodiments, the fastening means 300 is incorporated into the surfaces of the shaping and marking part 100 so as to lock them together and symmetrically compress the biological or synthetic tissue 400 that will be used to construct the leaflet 402. The fastening means 300 can be incorporated into the surfaces of the shaping and marking part 100 by magnetizing both surfaces in opposite ways (positive-negative).

In yet other possible embodiments, a clamp is used as the fastening means 300.

Preferably, at least one of the surfaces of the shaping and marking part 100 has along the edge of its inner surface a material conductive of electricity, which, upon applying current to it, enables the automatic cutting of tissue in the form of a leaflet.

In another aspect of the invention, a second medical device is provided, comprising a mold capable of indicating the exact positions of the surgical sutures 500 that are used to implant the replacement leaflets 402.

In one possible embodiment, the mold of said second medical device 20A is a semi-ring for insertion or implantation, which consists of an arched surface comprising:

at least one first groove 202 arranged to indicate at least one position in which at least one surgical suture 502 must be placed to fix the device 20A to a wall of an artery 404; and second grooves 204 arranged to indicate positions in which the surgical sutures 500 previously passed through the positions indicated by the first grooves 102 of the first medical device 10 must be placed.

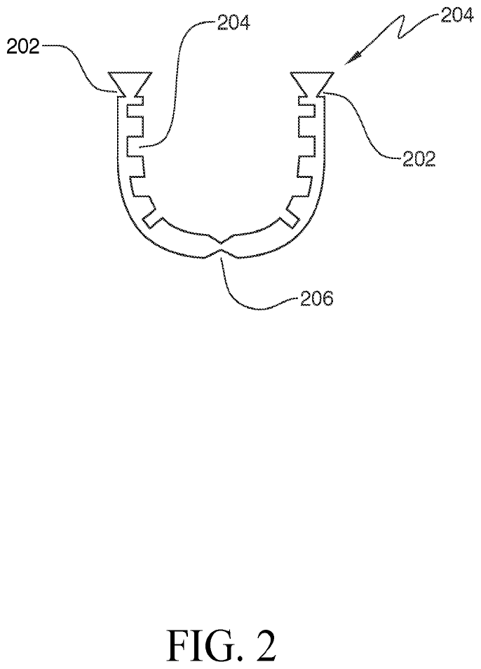

Preferably, the mold of said second medical device 20A is an arched surface in the shape of the letter “U”, as illustrated in FIG. 2.

Additionally, the arched surface may be flat or concavely or convexly curved.

In embodiments, the mold of said second medical device 20A comprises at least three first grooves 202, two at the ends and one at its nadir. In other embodiments, the second medical device 20A comprises a single first groove 202 at the nadir position.

In embodiments of the invention, the second grooves 204 are located in positions corresponding to the commissures and adjacent margins of the leaflet 402. Preferably, said second grooves 204 are internal.

In turn, the attachment half-ring 20A may comprise at least a third groove 206 designed to be fractured and allow the broken parts of the device 20A to be removed without getting tangled in the sutures 500 as soon as the leaflet 402 has been placed in position and partially sutured within the artery 404. Preferably, it comprises at least one third groove 206 located at the nadir, which may be the same one used to fix the device 20A (first groove 202).

Alternatively, in other possible embodiments of the invention, the mold of the second medical device 20B takes a three-dimensional tubular form resembling a three-pointed crown 201. This hollow mold is designed to accommodate the circulation of a dye fluid within it. The mold is equipped with openings 203 to allow the dye fluid to be expelled, thus marking an artery 404 at the positions where the leaflets 402 are to be placed. The tips 201 of the mold correspond to the commissures between the different leaflets 402.

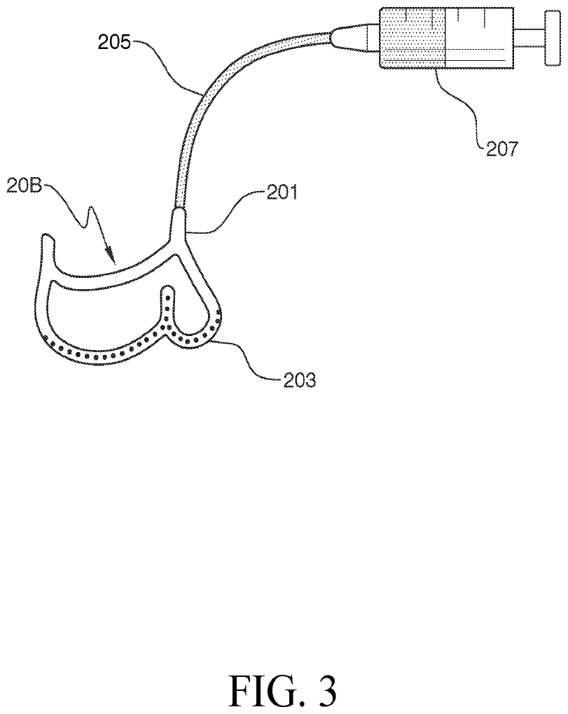

As depicted in FIG. 3, this three-dimensional tubular mold can be connected to a fluid distribution means, such as a hollow conduit 205. This conduit can be supplied, for instance, by a syringe 207 containing the dye fluid.

Consequently, in these embodiments, upon positioning the second medical device 20B within an artery 404 and infusing it with a dye fluid, it is possible to mark the interior of the said artery 404 at the precise locations where sutures should be positioned to anchor the leaflets 402 to its wall.

The size of the leaflet 402 and its position within the artery 404 are chosen based on the diameter of the patient's artery 404 (aorta or pulmonary artery, depending on the pathology) at the level of the sinotubular junction, preferably measured by echocardiography or computed tomography in the preoperative period. Therefore, the size of the first medical device 10 and the second medical device 20A or 20B will vary according to the size of the patient's native artery 404 to be treated.

In yet another aspect, a third medical device 30 is optionally provided, capable of simultaneously passing a plurality of sutures through the tissue. This device 30 consists of a stitching clamp comprising a first surface 31, at one of its tips, with fastening means (such as holes) suitable for receiving and holding needles. These fastening means are arranged at positions where the needles must penetrate the target tissue. The other tip of the third medical device 30 comprises a second surface 32 with at least one opening through which the needles pass upon exiting the tissue. Handles are present that are capable of bringing the tip surfaces 31 and 32 closer.

In certain embodiments of the invention, the third medical device 30 is used to pass surgical sutures anchored in leaflets 402 through an artery 404. In such cases, the fastening means on the first surface 31 receive and secure the needles corresponding to at least one commissure of a leaflet 402 that had previously gone through the tissue in the first medical device 10. These fastening means are positioned at the precise locations where the needles must penetrate the artery 404.

Preferably, the groove of the second surface 32 may contain a sheet of suture reinforcement material, such as Teflon Felt, to enable tying the sutures without damaging the wall of the artery 404.

The first surface 31 is positioned within the artery 404, and the second surface 32 is positioned outside, respecting the markings made by the second medical device 20A or 20B. By squeezing the clamp, the needles simultaneously pass through the wall of the artery 404 and exit through the second surface 32, also piercing, if present, the reinforcement material.

In certain embodiments, the third medical device 30 anchors only the sutures of one commissure of a leaflet 402, which results in the need to repeat the process six times (two for each of the three leaflets 402) to implant the whole valve.

In other embodiments, the third medical device 30 anchors simultaneously the sutures of both commissures of a leaflet 402, requiring the process to be repeated only three times (once for each leaflet 402) to implant the whole valve.

In yet other embodiments, the third medical device 30 anchors simultaneously the sutures of the commissures of adjacent leaflets 402, hence the process only needs to be repeated three times (once for each commissure of the valve) to implant the whole valve.

In alternative embodiments, the third medical device 30 is used to pass sutures through a replacement leaflet 402. In such cases, the fastening means that receive and secure the needles are arranged in the exact positions where they must penetrate the leaflet 402.

In still other embodiments, the third medical device 30 is used to suture leaflets 402 to an artery 404, where the distance between the fastening means that receive and secure the needles is adjustable to adapt the device 30 to pass sutures in both a replacement leaflet 402 and an artery 404.

In yet another aspect of the invention, a method of semilunar heart valve reconstruction is disclosed, comprising the steps of:
a) securing a portion of extended biological or synthetic tissue 400 with the first medical device 10, thereby firmly and tautly holding said tissue portion 400;
b) creating a replacement leaflet 402 from the tissue 400 by cutting around the shaping and marking part 100 of the first medical device 10, which has a leaflet-shaped configuration;
c) placing surgical sutures 500 on the replacement leaflet 402 at positions indicated by the first grooves 102 of the first medical device 10;
d) opening a patient's artery 404 and inserting the second medical device 20A or 20B;
e) passing the sutures 500 previously passed through the replacement leaflet 402 into positions indicated by the first grooves 102 of the first medical device 10 through the artery 404 in the positions indicated by the second medical device 20A or 20B;
f) suturing the nadir of the leaflet 402 to the artery wall to secure it;
g) removing the first medical device 10;
h) tying the sutures 500;
i) implanting a second and third replacement leaflets 402 by repeating steps a) to h).

The removal of the first medical device 10 can be carried out by breaking it through the second grooves 108 made for this purpose.

In preferred embodiments of the invention, tying the sutures in steps h) and j) involves tying the sutures 500 that have passed through the artery 404 outside of it and completing the leaflet implantation using running suture technique from the center towards the previously tied sutures.

In one possible embodiment of the invention, when in step d) the second medical device 20A is placed: it is secured to the artery 404 by placing sutures 502 through at least one position indicated by at least one first groove 202 of the second medical device 20A; in step g) the suture 502 that secures the second medical device 20A is additionally cut and removed; and the third replacement leaflet 402 is implanted by repeating at least steps a) to c), and then suturing the sutures 500 previously passed through the positions indicated by the first grooves 102 of the first medical device 10 to the artery 404, removing the first medical device 10, tying the sutures 500 outside the artery 404, and completing the leaflet implantation.

The removal of the second medical device 20A can be carried out by breaking it through the third grooves 206 made for this purpose.

Furthermore, the implantation of the third replacement leaflet 402 can be carried out by repeating steps a) to h).

According to another embodiment of the invention, when in step d) the second medical device 20B is placed, this step additionally involves introducing dye fluid into the second medical device 20B so as to mark the interior of the artery 404 at positions where the sutures of the leaflets 402 must be placed.

Optionally, after step c), the method may further comprise the step of anchoring the needles that passed through the replacement leaflet 402 in the holes of the third medical device 30. Step e) is performed by placing the third medical device 30 with the first surface 31 inside the artery 404 and the second surface 32 outside, then squeezing the handles so that the needles anchored in the first surface 31 simultaneously pass through the wall of the artery 404 and the second surface 32.

Optionally, step c) of the method is carried out using the third medical device 30, with the shaping and marking part 100 of the first medical device 10 positioned between the first 31 and second 32 surfaces of the third medical device 30. This is done by aligning the positions of the holes on the first surface 31 of the third medical device 30 with the first grooves 102 of the first medical device 10, and squeezing the handles so that the needles anchored in the first surface 31 simultaneously pass through the replacement leaflet 402.

The steps of the method will be detailed below according to preferred embodiments of the present invention.

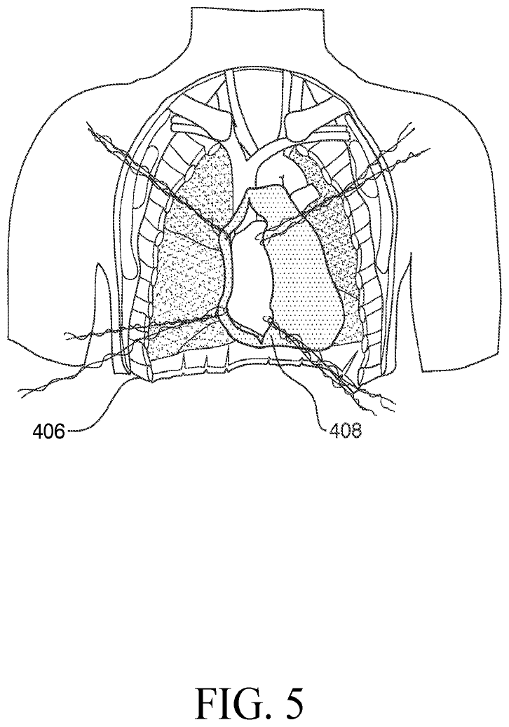

In a first stage of the proposed method, as illustrated in FIG. 5, after opening the chest 406, the pericardial sac 408 is opened in a cephalocaudal direction in the proximities of the phrenic nerve (preferably, the right one).

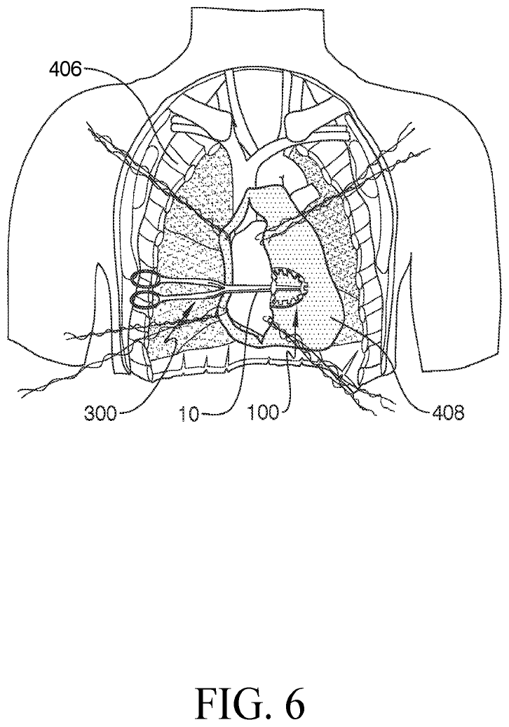

At the moment in which the leaflets 402 need to be made, a portion of the extended pericardium 400 is held with the first medical device 10, thus leaving a portion firmly trapped and stretched, as shown in FIG. 6.

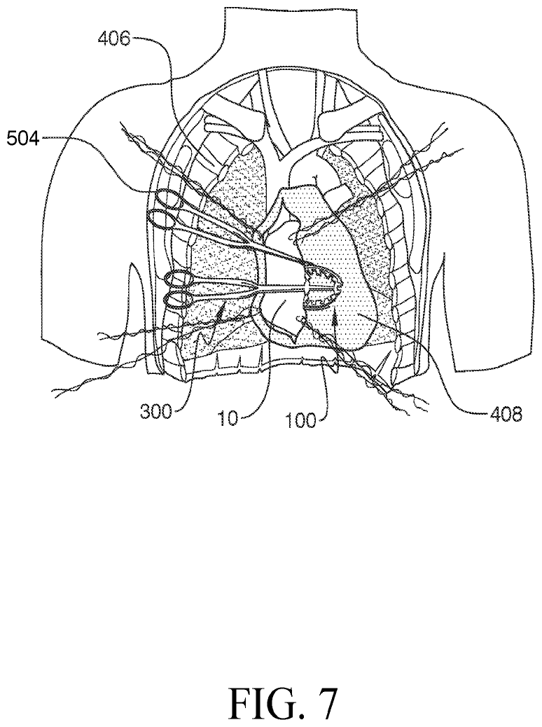

Once the biological tissue 400 is secured with the first medical device 10, the replacement leaflet 402 is cut around the shaping and marking part 100 that has its shape, as illustrated in FIG. 7.

This cut can be made just before the replacement leaflet 402 needs to be placed. This is especially useful when fresh autologous pericardium 400 is used, in order to keep it without irrigation for as short a time as possible.

The cutting can be performed at least partially automatically, such as in cases where the first medical device 10 is designed to perform the cut through an electrically conductive wire that can be powered by an electrosurgical unit, for example, or manually, using a scalpel or scissors. Preferably, the free edge 106 of the shaping and marking part 100 will coincide with the edge of the pericardium 400.

The sutures 500 are then passed through the replacement leaflet 402 at the positions indicated by the first grooves 102. This can be done manually, stitch by stitch, or by using the third medical device 30 to pass the sutures 500 simultaneously.

In the first internal grooves 102, which correspond to the commissures and adjacent margins of the leaflet 402, surgical sutures 500 are used in the shape of the letter “U”—with a needle at each end—. In the lower central groove 102 of the shaping and marking part 100, the same type of suture 500 is used to attach the leaflet 402 to the artery 404 wall and then complete the implantation using the running suture technique.

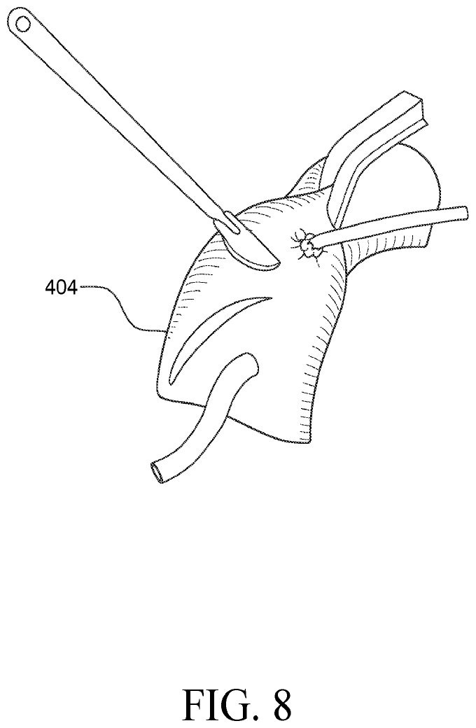

In a later stage of the method, as shown in FIG. 8, the patient's artery 404 is opened.

Subsequently, the second medical device 20A or 20B is used to mark the positions in the artery 404 where the surgical sutures 500 must pass through.

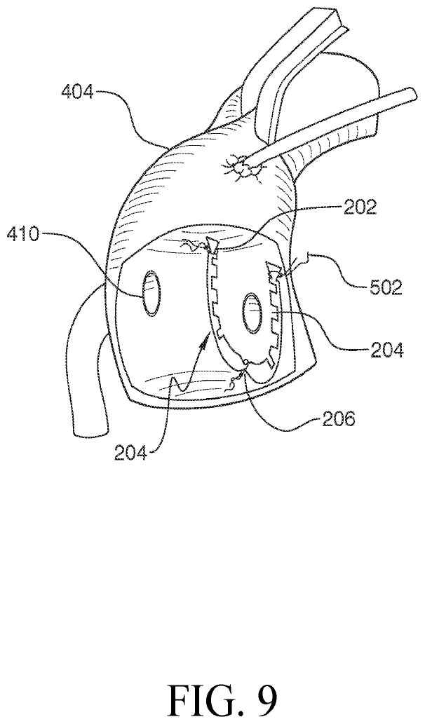

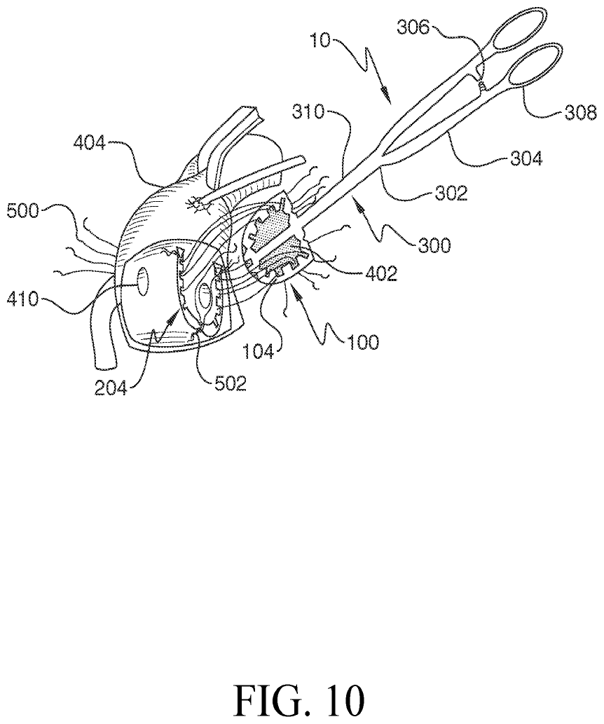

When the second medical device 20A is used, it is placed inside the artery 404, as depicted in FIG. 9, and anchored to the wall using fine sutures passed through the first grooves 202. The exact location of the device 20A is established in such a way that it does not obstruct the coronary ostia 410. Subsequently, as illustrated in FIG. 10, U-stitches 500 that have already been passed through the commissures and adjacent margins of the replacement leaflet 402 are passed through the artery 404 wall at the positions indicated by the second grooves 204.

On the contrary, when the second medical device 20B is used, it is placed inside the artery 404 and infused with a dye fluid, thus marking it at the positions where the sutures 500 that will anchor the leaflets 402 will be placed. Subsequently, the U-stitches 500 that had already been passed through the commissures and adjacent areas of the replacement leaflet 402 are now passed through the positions indicated by the dye fluid.

The process of passing the sutures 500 can be done manually, stitch by stitch, or by using the third medical device 30 to pass at least a plurality of sutures 500 simultaneously.

The nadir of the leaflet 402 is then sutured to the artery 404 wall to secure it. Both needles of this suture 500 come out of the sinus of Valsalva, that is, between the leaflet 402 and the artery 404 wall.

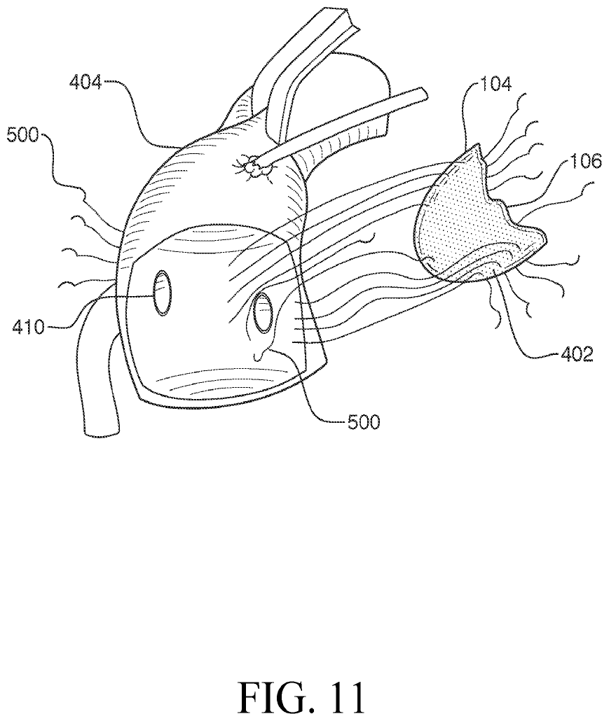

Proceeding with the method, the stitches 502 that attached the second medical device 20A (in case it was used) to the artery 404 wall are cut. The device 20A is removed, preferably fracturing it through the central groove 206 designed for this purpose. The shaping and marking part 100 of the first medical device 10 is also removed, preferably by fracturing it through the second grooves 108 provided for this purpose, leaving the leaflet 402 free to be approximated towards the artery 404 by pulling the sutures 500. FIG. 11 illustrates the leaflet 402 and the artery 404 after the first 10 and second 20 medical devices have been removed.

The order in which the devices are removed is indistinct.

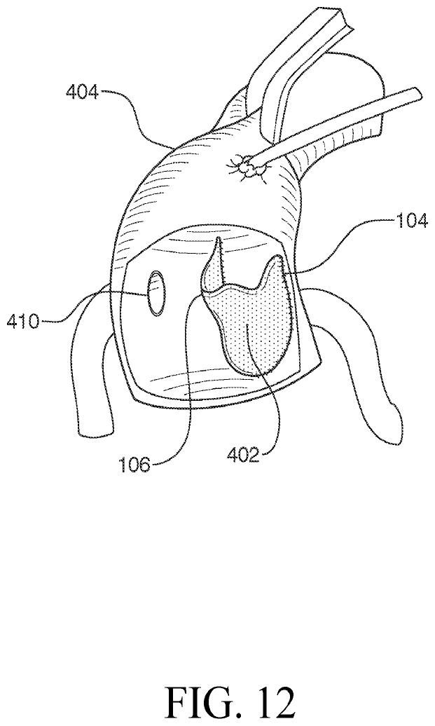

Subsequently, the U-stitches 500 are tied on the outer surface of the artery 404. The nadir suture 500 is then used to complete the implantation of the leaflet 402 using a running suture technique. FIG. 12 illustrates the artery 404 with the leaflet 402 already implanted.

The next two leaflets 402 are implanted in a similar way, repeating the aforementioned procedure.

In the case of the third leaflet 402, the use of the second medical device 20 may not be necessary, since the commissures of the adjacent leaflets 402 could be used as a reference.

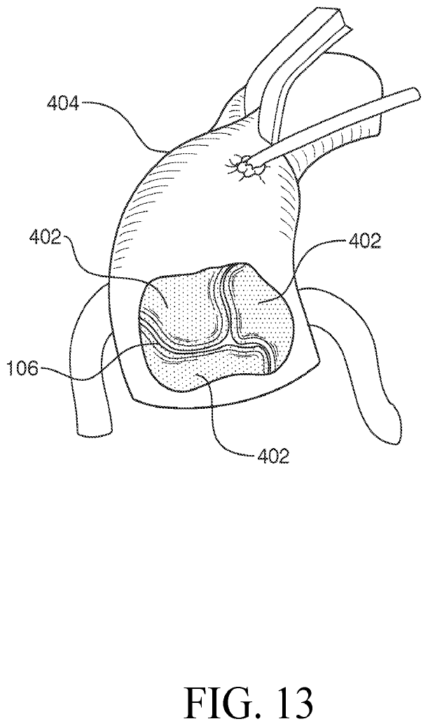

FIG. 13 illustrates the patient's artery 404 with all three replacement leaflets 402 in place.

For use in children, the size of the shaping and marking part 100 takes into account the patient's expected growth rate, in such a way that the resulting replacement leaflet 402 is oversized relative to the size of the artery 404. The second medical device 20A or 20B, unlike the first 10, is not oversized, that is, it has a size corresponding to the diameter of the native artery 404.

The reason behind the oversizing of the leaflets 402 is that the valve can continue to function properly as the child grows and the artery 404 increases in size.

A first feature of this oversizing is that the replaced valve has a windmill shape.

A second feature is that the coaptation zone, that is, the zone of apposition between leaflets 402, is increased. The coaptation zone is higher than normal to allow the valve to remain competent despite the reduction in coaptation height that occurs as the diameter of the artery 404 increases.

A third feature is a downward elongation of the leaflet 402 below the annular plane (also known as valve prolapse) when the oversizing is marked.

Unlike prior art methods, in the method of the present invention a coordinated replacement of the three leaflets 402 is carried out, completely removing the native valve and always obtaining a symmetrical valve with three identical leaflets 402 that meet in the center of the artery 404 during valve closure. The three sinuses of Valsalva that are generated are then equal in size, causing an equal distribution of pressure during diastole.

The method and medical devices described in this invention for the reconstruction of a semilunar heart valve contribute to the automation and the efficiency of the surgical procedure, thus reducing its duration and minimizing possible risks. In addition, they have other advantages: a) they can be used in adult and pediatric patients (children), b) they allow valve replacement with autologous tissue 400, c) they allow the replacement of native valves of any characteristic and number of leaflets, always achieving a symmetrical trileaflet valve, d) there is no need for anticoagulation after the procedure, and e) the new valve does not stop annulus growth and does not interfere with future valve replacement.

Finally, it is clear that the present invention is not limited to the construction exactly as illustrated and described; various modifications may be made, without changing the spirit of the scope of the present description. Consequently, when put into practice, modifications and/or improvements may be introduced, all of which must be considered as embodiment variants included within the scope of protection of this patent application.

1. A method for reconstructing a semilunar heart valve, comprising the following steps:
a) securing a portion of biological or synthetic tissue 400, which is extended using a first medical device 10, thereby firmly and tautly holding said portion of tissue 400, said first medical device 10 comprising a shaping and marking part 100 comprising two surfaces with an exterior configuration resembling that of a semilunar valve leaflet, designed to shape biological or synthetic tissue 400, said first surfaces having first grooves 102 arranged to mark positions at which surgical sutures 500 should be placed on the leaflet 402; and a fastening means 300 for the shaping and marking part 100 configured to maintain it in place, securing the tissue 400 in a stretched and immobilized manner between the two surfaces;
b) generating a replacement leaflet 402 from the tissue 400 by cutting it around the shaping and marking part 100 of the first medical device 10, which has a semilunar valve leaflet-shaped configuration;
c) placing sutures 500 in the replacement leaflet 402 at positions indicated by the first grooves 102 of the first medical device 10;
d) opening a patient's artery 404 and inserting a second medical device 20A or 20B therein, said second medical device 20A or 20B comprising a mold capable of indicating positions for sutures 500 to be placed in order to insert the replacement leaflet 402;
e) passing the sutures 500, previously passed through the replacement leaflet 402 at positions indicated by the first grooves 102 of the first medical device 10, through positions indicated by the second medical device 20A or 20B, passing through an artery 404 wall;
f) suturing the nadir of the replacement leaflet 402 to the wall of the artery 404 to secure it;
g) removing the first medical device 10;
h) tying the sutures 500;
i) implanting a second and third replacement leaflet 402 by repeating steps a) to h).

2.-4. (canceled)

5. The method of claim 1, wherein at least one of the surfaces of the shaping and marking part 100 of the first medical device 10 has a roughness or fine teeth on at least one of its faces arranged to help hold the biological or synthetic tissue when exerting pressure by means of the fastening means 300.

6.-9. (canceled)

10. The method of claim 1, wherein the surfaces of the shaping and marking part 100 of the first medical device 10 additionally have second grooves 108 designed to facilitate fracture and removal once the leaflet 402 has been positioned and partially sutured within an artery 404.

11. (canceled)

12. The method of claim 1, wherein said mold of the second medical device 20A is an arched surface comprising:
at least one first groove 202 arranged to indicate at least one position for placing at least one suture 502 that secures the device 20A to an artery 404 wall; and
second grooves 204 arranged to indicate positions where the sutures 500, previously passed through the positions indicated by the first grooves 102 of the first medical device 10, should be placed.

13.-19. (canceled)

20. The method of claim 1, wherein said mold of the second medical device 20B is a three-pointed 201 tubular three-dimensional mold in the shape of a crown that corresponds to the commissures between the different leaflets 402, the mold being hollow to allow the circulation of a dye solution inside it, and featuring perforations 203 to enable the removal of the dye solution, thereby marking an artery 404 at positions where the leaflets 402 should be placed.

21. The method of claim 20, wherein said mold is connectable to a fluid distribution means 205 that can be fed by a syringe 207 containing the dye solution.

22. The method of claim 20, wherein in step d), the second medical device 20B is positioned, and additionally, this step involves feeding the second medical device 20B with a dye solution, thereby marking the interior of the artery 404 at positions where the sutures 500 of the leaflets 402 should be placed.

23. The method of claim 1, wherein tying the sutures 500 in steps h) and j) involves tying the sutures that passed through the artery 404 outside of it and completing the leaflet implantation with the suture 500 positioned at the nadir using running suture technique from the center toward the previously tied sutures.

24. The method of claim 1, wherein after step c), the method comprises the step of anchoring the needles that passed through the replacement valve 402 in a fastening means of a third medical device 30, which consists of a stitching clamp comprising a first surface 31 at one of its ends with suitable fastening means to receive and secure needles, these fastening means being arranged in positions where the needles must pass through target tissue, a second surface 32 at the other end with at least one opening through which the needles exit the tissue, as well as handles capable of bringing the tip surfaces together, causing multiple sutures 500 to pass simultaneously through the target tissue; and step e) is carried out by placing the third medical device 30, positioning the first surface 31 within the artery 404 and the second surface 32 outside, and squeezing its handles so that the needles anchored in the first surface 31 simultaneously pass through the artery 404 wall and the second surface 32.

25. (canceled)

26. The method of claim 1, wherein step c) is carried out using a third medical device 30 that consists of a stitching clamp comprising a first surface 31 at one of its ends with suitable fastening means to receive and secure needles, said fastening means being arranged in positions where the needles must pass through the target tissue, a second surface 32 at the other end with at least one opening through which the needles exit the tissue, as well as handles capable of bringing the tip surfaces together, causing multiple sutures to pass simultaneously through the target tissue, positioning the shaping and marking part 100 of the first medical device 10 between the first and second surfaces of the third medical device 30, aligning the positions of the fastening means of the first surface 31 of the third medical device 30 with the first grooves 102 of the first medical device 100, and squeezing the handles so that the needles anchored in the first surface 31 simultaneously pass through the replacement leaflet 402.

27. A first medical device 10 applicable to the method for reconstructing semilunar heart valves of claim 1, comprising: a shaping and marking part 100 comprising two leaflet-shaped surfaces designed to mold biological or synthetic tissue 400, wherein said surfaces have first grooves 102 arranged to indicate positions for suture 500 placement on the leaflet 402; and a fastening means 300 for the shaping and marking part 100 configured to maintain it in place, securing the tissue 400 in a stretched and immobilized manner between the two surfaces.

28.-30. (canceled)

31. The first medical device 10 of claim 27, wherein at least one of the surfaces of the shaping and marking part 100 has a roughness or fine teeth on at least one of its faces, arranged to assist in holding the tissue 400 by exerting pressure through the fastening means 300.

32.-35. (canceled)

36. The first medical device 10 of claim 27, wherein the surfaces of the shaping and marking part 100 additionally have second grooves 108 formed to facilitate fracture and removal once the leaflet 402 has been positioned and partially sutured within an artery 404.

37. (canceled)

38. A second medical device 20A or 20B applicable to the method for reconstructing semilunar heart valves of claim 1, comprising a mold capable of indicating positions for sutures 500 to be placed for the implantation of replacement leaflets 402.

39. The second medical device 20A of claim 38, wherein said mold is an arched surface comprising:
at least one first groove 202 arranged to indicate at least one position for placing at least one suture 502 that secures the device 20A to an artery 404 wall; and
second grooves 204 arranged to indicate positions where sutures 500, previously passed through the leaflet 402 at the positions indicated by the first grooves 102 of the first medical device 10, should be placed.

40.-43. (canceled)

44. The second medical device 20A of claim 39, wherein said mold additionally comprises at least one third groove 206 designed to facilitate its fracture and removal once a leaflet 402 has been positioned and partially sutured within an artery 404.

45. (canceled)

46. The second medical device 20B of claim 38, wherein said mold is a three-dimensional tubular mold in the shape of a three-pointed crown 201 that corresponds to the commissures between the different leaflets 402, the mold being hollow to allow the circulation of a dye solution inside it and having perforations 203 to enable the removal of the dye solution, thereby marking an artery 404 at positions where the leaflets 402 should be placed.

47. The second medical device 20B of claim 46, wherein said mold is connectable to a fluid distribution means 205 that can be fed by a syringe 207 containing the dye solution.

48. A third medical device 30 applicable to the method for reconstructing semilunar heart valves of claim 24 wherein said device 30 consists of a stitching clamp comprising a first surface 31 at one of its ends with suitable fastening means to receive and secure needles, said fastening means being arranged in positions where the needles must pass through the target tissue, a second surface 32 at the other end with at least one opening through which the needles exit the tissue, as well as handles capable of bringing the tip surfaces together, causing multiple sutures to pass simultaneously through the target tissue.

49. (canceled)

50. A third medical device 30 applicable to the method for reconstructing semilunar heart valves of claim 26, wherein said device 30 consists of a stitching clamp comprising a first surface 31 at one of its ends with suitable fastening means to receive and secure needles, said fastening means being arranged in positions where the needles must pass through the target tissue, a second surface 32 at the other end with at least one opening through which the needles exit the tissue, as well as handles capable of bringing the tip surfaces together, causing multiple sutures to pass simultaneously through the target tissue.
